(19) United States
(12) Patent Application Publication
(10) Pub. No.: US 2011/0021769 A1
(43) Pub. Date: Jan. 27, 2011

(54) Process for Producing Fluorocytidine Derivatives

(75) Inventors: Tsung-Cheng Hu, Tongjing Township (TW); Hong-Tsung Huang, Taisi Township (TW)

Correspondence Address: COHEN, PONTANI, LIEBERMAN & PAVANE LLP, 551 FIFTH AVENUE, SUITE 1210, NEW YORK, NY, 10176, US

(73) Assignee: ScinoPharm Taiwan Ltd.

(21) Appl. No.: 12/840,490

(22) Filed: Jul. 21, 2010

Related U.S. Application Data
(60) Provisional application No. 61/227,971, filed on Jul. 23, 2009.

(51) Int. Cl. C07H 19/067

(52) U.S. Cl. 536/28.51

(57) ABSTRACT

A process for making a capecitabine or its derivative comprising (a) reacting a compound of the formula (II):

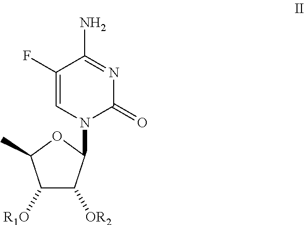

wherein each of R₁ and R₂ independently represents a hydroxyl protecting group, with an acylating agent of formula (III): X—C(═O)—R₃, wherein X is an acyl activating group in an organic solvent to produce an acylated compound; (b) deprotecting the acylated compound to obtain the compound of formula (I); and (c) purifying the compound of formula (I) with a solvent.

### RELATED APPLICATIONS

This application claims priority from U.S. Provisional Patent Application Ser. No. 61/227,971 which was filed on Jul. 23, 2009. The entire content of this provisional application is incorporated herein as reference.

### BACKGROUND OF THE INVENTION

1. Field of Invention

The present application relates to a process for manufacture of 5′-deoxy-5-fluoro-N⁴-n-pentyloxycarbonylcytidine (capecitabine) and its derivatives.

2. Description of the Related Art

Capecitabine is a fluoropyrimidine carbamate with antineoplastic activity and is commercially available in the market under the brand name XELODA®, having the following chemical structure:

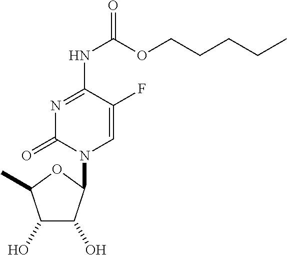

The synthesis of capecitabine is described in several publications including U.S. Pat. Nos. 5,472,949; 4,966,891; 5,453,497; 7,365,188; and 5,476,932.

However, there is still a need for an improved process of making capecitabine and its derivatives.

### SUMMARY OF THE INVENTION

One aspect of the present application provides a process of making a purified compound of formula (I):

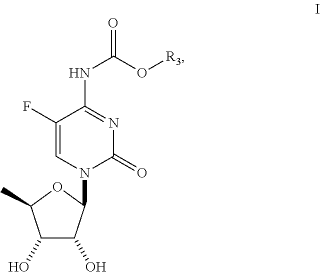

wherein R₃ is alkyl, cycloalkyl, aralkyl, aryl, or alkoxy, preferably C1˜C12 alkyl, cycloalkyl, aralkyl, aryl, or alkoxy, and more preferably C1˜C6 alkyl. The process comprises:

(a) reacting a compound of the formula (II):

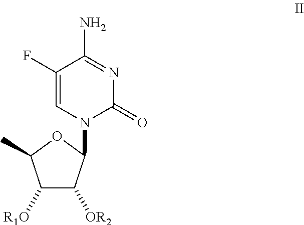

wherein each of R₁ and R₂ independently represents a hydroxyl protecting group, with an acylating agent of formula (III): X—C(═O)—R₃, wherein X is an acyl activating group, R₃ is as defined above, in an organic solvent, such as CH₂Cl₂, THF, acetonitrile, toluene, or ethyl acetate, to produce an acylated compound of formula (IV):

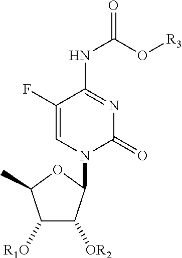

wherein each of R₁, R₂, and R₃ is as defined above;
(b) deprotecting the acylated compound of formula (IV) to obtain the compound of formula (I); and
(c) purifying the compound of formula (I) with a solvent.

Preferably, the hydroxyl protecting group is acetyl or benzoyl.

X in the above acylating agent of formula (III) is preferably halide, more preferably chloride. The acylating agent of formula (III) is preferably n-pentyl chloroformate.

The compound of formula (I) is preferably capecitabine, i.e., R₃ in the above formula (I) is a pentyl group.

The reacting step (a) in the above process is preferably carried out in the presence of a base. The base is preferably in an amount from 3.5 to 5.0, more particularly about 4.0 mole equivalents of the compound of formula (II). The base is preferably pyridine.

The deprotecting step (b) in the above process is preferably carried out in the presence of a base. The base is preferably sodium hydroxide. As a preferred embodiment, the deprotecting step (b) is accomplished by a hydrolysis reaction in a temperature of from about 0 to 10° C., more particularly from about 0 to 5° C.

As a preferred embodiment, the reacting step (a) and deprotecting step (b) are successively carried out in the same reactor. In other words, the process of the present application may be carried out in one pot.

The process as described above does not comprise a step of silylating the compound of formula (II) or any compound coupled by a 5-fluorocytosine or its derivative with a 5-deoxy furanoside or its derivative.

The purifying step c) of the above process is preferably carried out at a temperature of less than 60° C. The solvent used in the purifying step may be water, ketone, ester (such as ethyl acetate), alcohol, ether, and combinations thereof. For example, the solvent may be water, n-pentanol, a mixture of n-pentanol and n-heptane, and a mixture of ethyl acetate and n-heptane. In particular, the purifying step comprises crystallizing the compound of formula (I) from n-pentanol alone or a mixture of n-pentanol with one or more other solvents.

Another aspect of the present application provides capecitabine having the following mean particle size distribution:

D₉₀: 250 to 350 microns, D₅₀: 100 to 120 microns, and D₁₀: 25 to 30 microns.

Yet another aspect of the present application provides a process of making capecitabine. The process comprises deprotecting a compound of formula (IV)

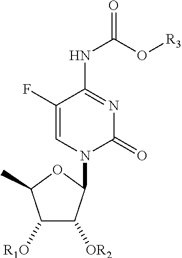

### IV

with an enzyme, wherein each of R₁ and R₂ independently represents a hydroxyl protecting group, R₃ is alkyl, cycloalkyl, aralkyl, aryl, or alkoxy, preferably C₁˜C₁₂ alkyl, cycloalkyl, aralkyl, aryl, or alkoxy, more preferably, C₁˜C6 alkyl. Preferably, R₁ and R₂ both represent the same hydroxyl protecting group, such as acetyl and benzoyl.

Preferably, the enzyme is lipase. The reaction temperature is preferably from 20 to 60° C. The reaction pH range is preferably from 4 to 9. R₃ is preferably a pentyl group.

The enzyme may deprotect the 2′ and 3′ position protecting groups with high specificity. In addition, enzymatic hydrolysis may be carried out in mild condition, and the enzyme may be used repeatedly.

Another aspect of the present application provides a capecitabine comprising:
no more than 0.3% by HPLC area percent (A %) of impurity F

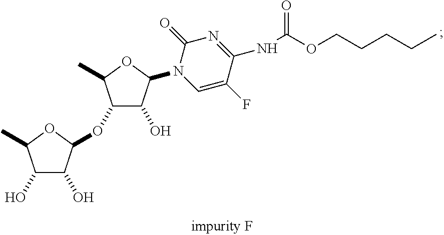

no more than 0.2% by HPLC area percent (A %) of impurity G,

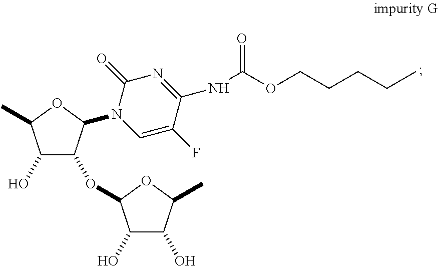

no more than 0.3% by HPLC area percent (A %) of impurity H,

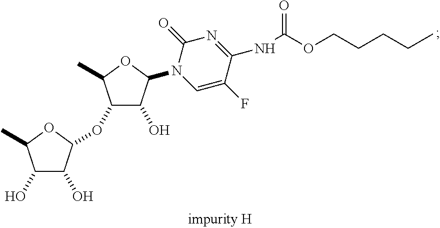

no more than 0.1% by HPLC area percent (A %) of M2,

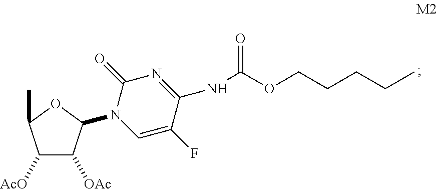

and
no more than 0.10% by HPLC area percent (A %) of impurity M

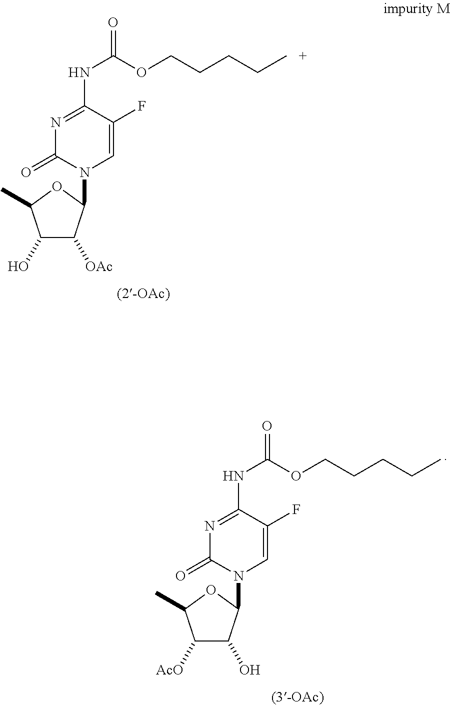

Therefore, the present application provides an improved process for industrial scale and a facile final purification of the compound of formula (I), in particular capecitabine, with high purity (>99.5%) and less undesired alpha-form impurity.

The various features of novelty which characterize the invention are pointed out with particularity in the claims annexed to and forming a part of the disclosure. For a better understanding of the invention, its operating advantages, and specific objects attained by its use, reference should be had to the descriptive matter in which there are illustrated and described preferred embodiments of the invention.

### BRIEF DESCRIPTION OF THE DRAWINGS

In the drawings:

FIG. 1 shows appearance of capecitabine products obtained in accordance with Example 5 of the present application.

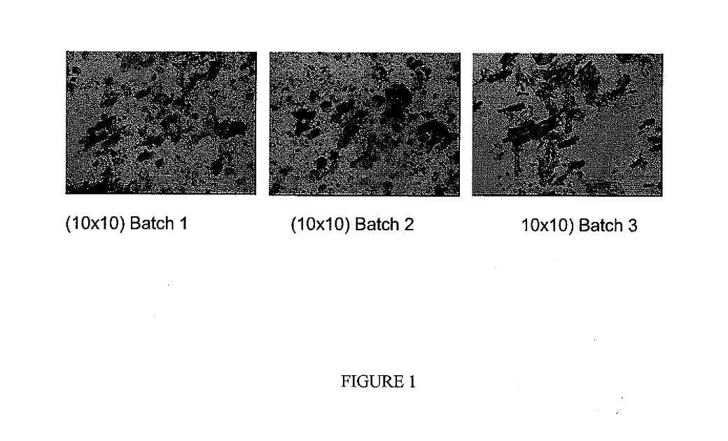

### DETAILED DESCRIPTION OF THE PRESENTLY PREFERRED EMBODIMENTS

The following preferred embodiments are provided for further illustrating, but not for limiting, the present invention.

In accordance with one embodiment of the present application, the processes of making capecitabine may be illustrated by the following scheme:

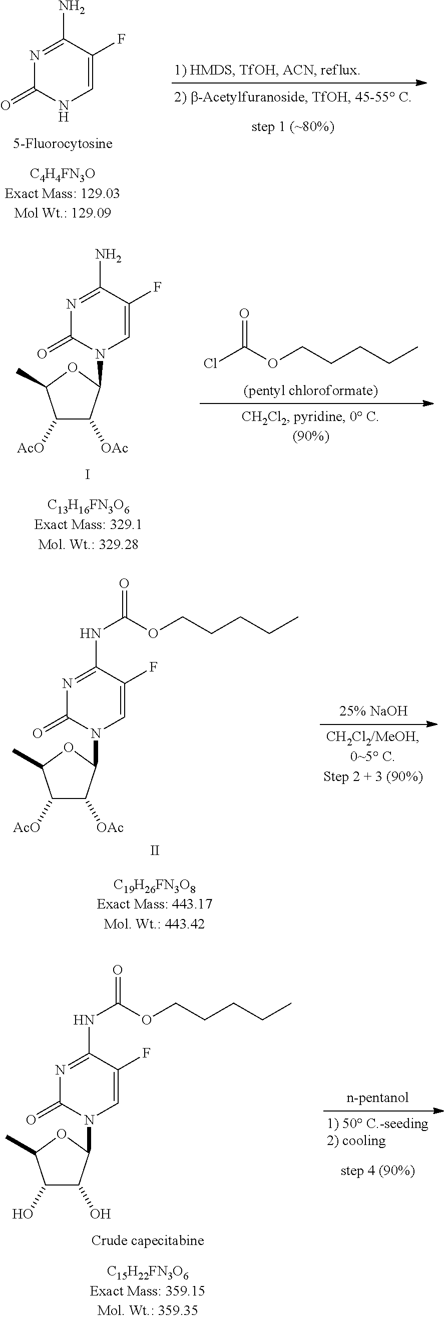

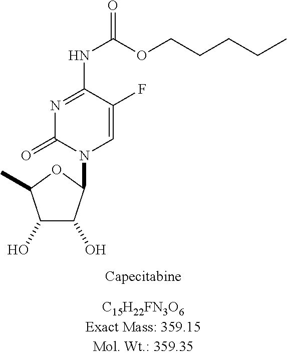

After completion of the reaction, the crude capecitabine can be purified under water system. The purity of capecitabine is 99.4% (by HPLC area percent (A %)), impurity F≦0.3%, impurity G≦0.2%, impurity H≦0.3%, M2≦0.1%, impurity M≦0.10% and the maximum individual impurity is ≦0.1%. Unless explicitly stated otherwise, the purities discussed in this application are all based on HPLC area percent (A %).

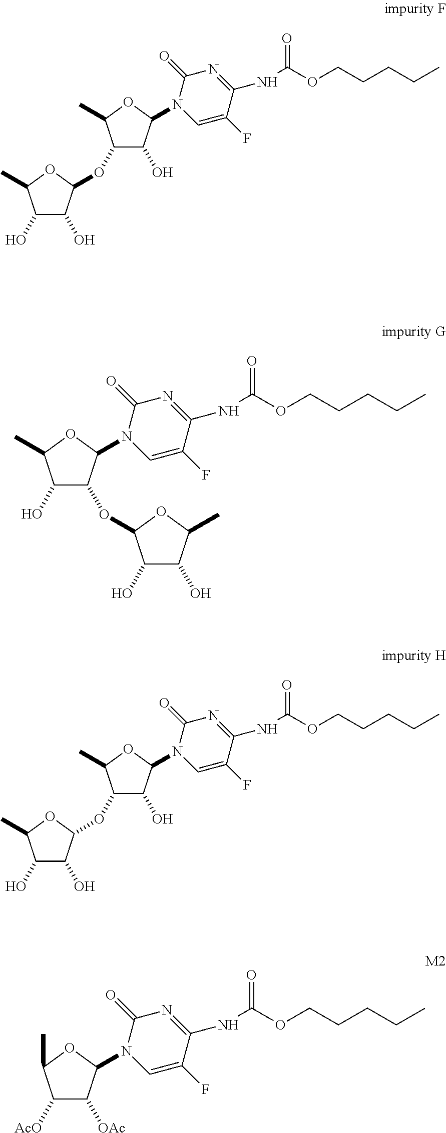

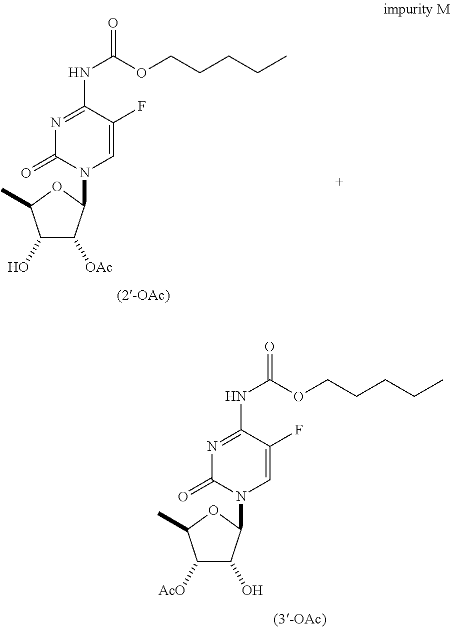

After completion of the reaction, the crude capecitabine may be purified under ethyl acetate system. The purity of capecitabine is ≧99.5%, impurity F≦0.3%, impurity G≦0.2%, impurity H≦0.3%, M2≦0.1%, impurity M≦0.10% and the maximum individual impurity is ≦0.1%.

In another embodiment, the inventors of this invention have developed a novel process for deprotection of protecting groups of capecitabine selectively with enzyme. Enzymatic hydrolysis can be carried out in mild condition and the enzyme may be used repeatedly. In addition, enzymatic hydrolysis reaction can avoid the side products and other impurities produced during the deprotection step.

The enzymatic hydrolysis reaction comprises treating a compound of formula (IV′) with enzyme to selectively deacylate the 2′ and 3′ positions of the carbohydrate moiety to produce capecitabine.

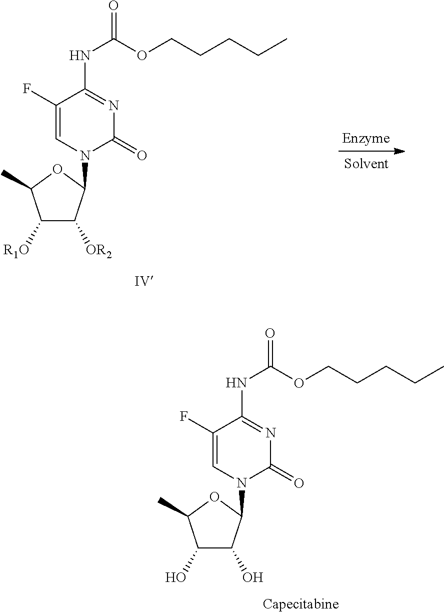

wherein each of R₁ and R₂ is independently a hydroxyl protecting group. Preferably, R₁=R₂=acetyl or benzoyl.

As a specific embodiment, the process of the application may be illustrated by the following scheme:

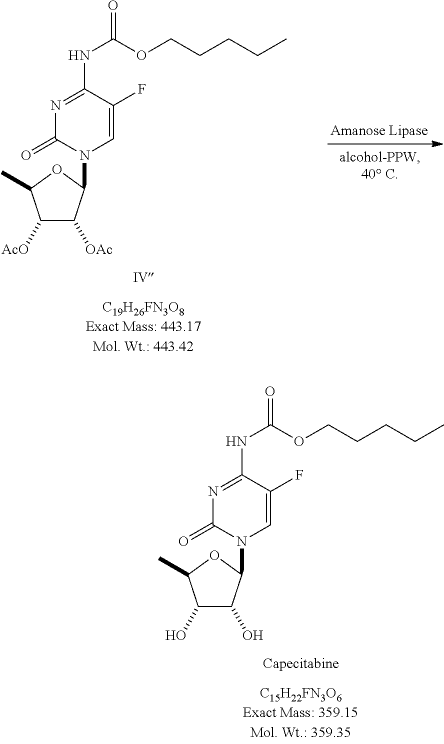

The following examples are provide to further illustrating, but by no means for limiting, the present invention.

### EXAMPLES

### Example 1

### A Process for Producing and Purification of 2′,3′-di-O-acetyl-5′-deoxy-5-fluorocytidine (I)

To a vessel is added of 5-fluorocytosine (1.2 kg, 9.30 mol), triflic acid (5.0 g), hexamethyldisilazane (1.06 kg, 6.57 mol) and acetonitrile (4.3 kg). The mixture is heated to reflux and keeps reflux for about 2 hours. The solution is cooled to room temperature and added of β-acetylfuranoside (2.528 kg, 9.71 mol) and triflic acid (0.832 kg, 5.54 mol). The resultant mixture is heated and stirred at 45-55° C. for about 20 hours. After completion of the reaction, the solution is cooled to 20-30° C. and worked up with saturated sodium bicarbonate solution. After phase separation with methylene chloride, the organic layer is collected and subsequently swapped with isopropanol (7.76 kg) to an appropriate volume. The resulting isopropanol solution is heated to reflux until dissolved. The solution is cloud after seeding with 2′,3′-di-O-acetyl-5′-deoxy-5-fluorocytidine at 50-70° C. The slurry is cooled to room temperature and n-heptane is charged with stirring for another 0.5 hrs. The solution is cooled to less than 10° C. The resulting solid is filtered, washed with cold isopropanol and dried under vacuum, to afford 2′,3′-di-O-acetyl-5′-deoxy-5-fluorocytidine. The purity is ≧99.5% and related alpha-form impurity is ≦0.2%. Yield: 80%. ¹H NMR (CDCl₃, 400 MHz) δ 7.85 (s, 1H), 7.84 (b, NH), 7.09 (b, NH), 5.87 (m, 1H), 5.50 (m, 1H), 5.17 (m, 1H), 4.15 (m, 1H), 2.07 (s, 6H), 1.43 (d, J=6.4 Hz, 3H).

### Example 2

### A process for producing and purification of 2′,3′-di-O-acetyl-5-deoxy-5-fluoro-N4-(pentyl-oxycarbonyl)cytidine (II)

To a vessel is added of 2′,3′-di-O-acetyl-5′-deoxy-5-fluorocytidine (0.2 kg, 0.6 mol), methylene chloride (1.59 Kg) and pyridine (190.0 g, 2.4 mol) at 20-30° C. The mixture is cooled to below 5° C. and subsequently is added of n-pentylchloroformate (137.2 g, 0.9 mol) at below 10° C. The resulting solution is stirred at less than 10° C. for at least 0.5 hour. After completion of the reaction, water (2 Kg) is added for phase separation. The organic layer is collected and washed with water (2 kg) for three times. Then organic layer is collected and swapped with toluene (0.4 Kg) under vacuum at less than 60° C. After solvent swap, n-heptane (0.3 kg) is added to cloud point at 40-50° C. After stirring at 40-50° C. for about 1 hour, n-heptane (0.4 kg) is added and the slurry is cooled to less than 10° C. The solution keeps stirring for at least 1 hour. The resulting solid is filtered, washed with toluene/n-heptane (1:9) and dried under vacuum to afford 2′,3′-di-O-acetyl-5-deoxy-5-fluoro-N4-(pentyl-oxycarbonyl)cytidine. The purity is ≧99.5% and the maximum impurity is ≦0.2%. Yield: ¹H NMR (CDCl₃, 400 MHz) δ 8.05 (d, J=6.4 Hz, 1H), 5.93 (m, 1H), 5.52 (m, 1H), 5.15 (m, 1H), 4.24 (m, 1H), 4.15 (m, 2H), 2.06 (s, 6H), 1.68 (m, 2H), 1.47 (d, J=6.4 Hz, 3H), 1.38 (m, 4H), 0.91 (m, 3H).

### Example 3

### A Process for Producing and Purification of Capecitabine Under Water System

To a vessel is added of 2′,3′-di-O-acetyl-5-deoxy-5-fluoro-N4-(pentyl-oxycarbonyl)cytidine (20 g, 45.1 mmol), methylene chloride (160 g) and methanol (20 mL) at below 5° C. Subsequently 25% NaOH (16 g, 100 mmol) is added at below 5° C. The resulting solution is maintained at below 5° C. and stirred for at least 0.5 hour. After completion of the reaction, citric acid (60 g) is added for quenching the reaction and doing phase separation. The organic layer is collected and the aqueous is continued to wash with methylene chloride (40 mL). After phase separation, the methylene chloride layer is collected and combined with the previous organic layer. The resulting organic layer is washed with water (100 g) and the organic layer is collected. The organic layer is concentrated and then is swapped with water (100 g) under vacuum at less than 60° C. After solvent swap, the resulting solution is heated at 40-55° C. and seeded with capecitabine. The mixture is held for about 1 hour at 20-55° C. and cooled to −5 to 5° C. The slurry is stirred at −5 to 5° C. for about 2 hours. The resulting solid is filtered, washed with cold water and dried under vacuum to afford capecitabine. The purity is ≧99.4%, impurity F≦0.3%, impurity G≦0.2%, impurity H≦0.3%, M≦0.1%, impurity M≦0.10% and the maximum individual impurity is ≦0.1%. Yield: 47%.

| Chemical Name | Chemical Structure | Product Name |
|---|---|---|
| [1-[5-deoxy- 3-O-(5-deoxy-β- d-ribofuranosyl)- β-d- ribofuranosyl]- 5-fluoro-2- oxo-1,2- dihydropyr-imidin-4- yl]-carbamic acid pentyl ester | 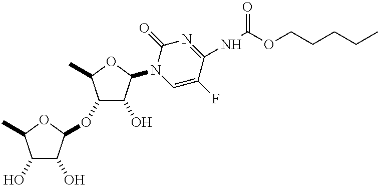 | impurity F |
| [1-[5-deoxy-2-O- (5-deoxy-β- d-ribofuranosyl)- β-d- ribofuranosyl]- 5-fluoro-2- oxo-1,2- dihydropyr-imidin-4- yl]-carbamic acid pentyl ester | 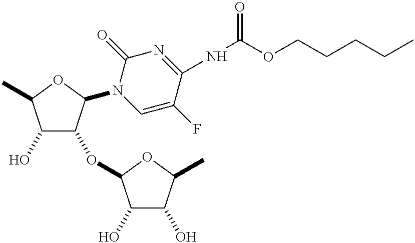 | impurity G |
| [1-[5-deoxy-3- O-(5-deoxy-α- d-ribofuranosyl)- β-d-ribo- furanosyl]- 5-fluoro-2- oxo-1,2- dihydropyr-imidin-4- yl]-carbamic acid pentyl ester | 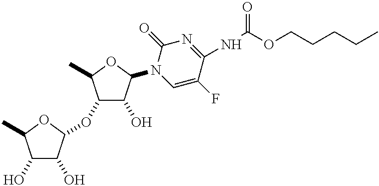 | impurity H |
| 2,3′-di-O-acetyl- 5-deoxy-5- fluoro- N4-(pentyl- oxycarbonyl)cytidine | 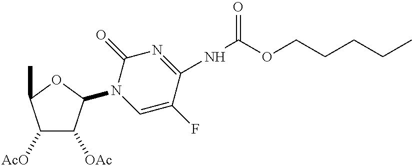 | M2 |
| 2-O-acetyl-5- deoxy-5-fluoro- N4-(pentyloxy- carbonyl)cytidine and 3-O-acetyl-5- deoxy-5-fluoro- N4-(pentyloxy- carbonyl)cytidine | 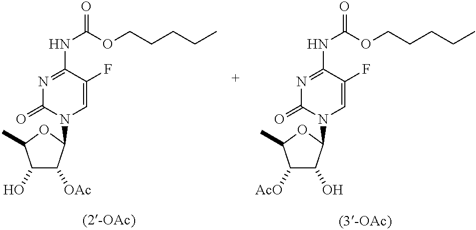 | impurity M |

### Example 4

### A Process for Producing and Purification of Capecitabine Under Ethyl Acetate System

To a vessel is added of 2′,3′-di-O-acetyl-5-deoxy-5-fluoro-N4-(pentyl-oxycarbonyl)cytidine (20 g, 45.1 mmol), methylene chloride (160 g) and methanol (20 mL) at below 5° C. Subsequently 25% NaOH (16 g, 100 mmol) is added at below 5° C. The resulting solution is maintained at below 5° C. and stirred for at least 0.5 hour. After the completion of the reaction, citric acid (60 g) is added for quenching the reaction and doing phase separation. The organic layer is collected and the aqueous is continued to wash with methylene chloride (40 mL). After phase separation, the methylene chloride layer is collected and combined with the previous organic layer. The resulting organic layer is washed with water (100 g) and the organic layer is collected. The organic layer is concentrated and then is swap with ethyl acetate (60 mL) under vacuum at less than 60° C. After solvent swap, n-heptane (20 mL) is added and the resulting solution is heated at 40-55° C. and seeded with capecitabine. The mixture is held for about 1 hour at 40-55° C. and cooled to −5 to 5° C. The slurry is stirred at −5 to 5° C. for about 2 hours. The resulting solid is filtered, washed with n-heptane and dried under vacuum to afford capecitabine. The purity is ≧99.5%, impurity F≦0.3%, impurity G≦0.2%, impurity H≦0.3%, M2≦0.1%, impurity M≦0.10% and the maximum individual impurity is ≦0.1%. Yield: 85%.

### Example 5

### A process for producing and purification of capecitabine from 2′,3′-di-O-acetyl-5′-deoxy-5-fluorocytidine in One-Pot reaction

To a vessel is added of 2′,3′-di-O-acetyl-5′-deoxy-5-fluorocytidine (31.5 kg, 95.6 mol), methylene chloride (230 kg) and pyridine (30 kg, 379.3 mol) at 20-30° C. The mixture is cooled down to below 5° C. and subsequently is added of n-pentylchloroformate (22 kg, 146.1 mol) at below 10° C. The resulting solution is stirred at less than 10° C. for at least 0.5 hour. After completion of the reaction, water (500 g) is added for phase separation. The organic layer is collected and washed with water (500 g) for about three times. Then organic layer is collected and transferred to a vessel. Then methanol (38.7 g) is added at below 5° C. Subsequently 25% NaOH (36 g, 0.22 mol) is added at below 5° C. The resulting solution is maintained at below 5° C. and stirred for at least 0.5 hour. After completion of the reaction, citric acid (135 g) is added for quenching the reaction and doing phase separation. The organic layer is collected and the aqueous is continued to wash with methylene chloride (112 g). After phase separation, the methylene chloride layer is collected and combined with the previous organic layer. The resulting organic layer is washed with water (225 g) and the organic layer is collected. The organic layer is concentrated and then is swapped with n-pentanol (225 mL) under vacuum at less than 60° C. After solvent swap, the resulting solution is heated at 40-55° C. and seeded with capecitabine. The mixture is held for about 1 hour at 40-55° C. and cooled down to −5 to 5° C. The slurry is stirred at −5 to 5° C. for about 2 hours. The resulting solid is filtered, washed with n-heptane and dried under vacuum to afford capecitabine. The purity is ≧99.5%, impurity F≦0.3%, impurity G≦0.2%, impurity H≦0.3%, M2≦0.1%, impurity M≦0.10% and the maximum individual impurity is ≦0.1%. Yield: 77%.

| | Sample batch | | |
|---|---|---|---|
| | Batch 1 | Batch 2 | Batch 3 |
| Solvent | n-pentanol and wash | n-pentanol and wash | n-pentanol and |
| | with n-heptane | with n-heptane | wash with |
| | | | n-heptane |
| Powder flow | Very Poor | Very Poor | Very Poor |
| Tapped | 0.3848 | 0.3654 | 0.3888 |
| density | | | |
| (g/ml) | | | |
| Bulk density | 0.1922 | 0.1808 | 0.2168 |
| (g/ml) | | | |
| Water | 0.0178 | 0.017 | 0.008 |
| content (%) | | | |
| PSD | 343.66 | 252.22 | 306.11 |
| (D₉₀, μm) | | | |
| PSD | 120.55 | 100.24 | 121.38 |
| (D₅₀, μm) | | | |
| PSD | 28.95 | 29.46 | 30.16 |
| (D₁₀, μm) | | | |

* D₉₀ is 90% less than; D₅₀ is 50% less than; D₁₀ is 10% less than.

### Example 6

### A Process for Producing and Purification of Capecitabine Under n-Pentanol and a Mixed Solvent System

To a vessel is added of 2′,3′-di-O-acetyl-5′-deoxy-5-fluorocytidine (1.0 kg, 3.0 mol), methylene chloride (7.0 kg) and pyridine (0.96 kg, 19.5 mol) at 20-30° C. The mixture is cooled to below 5° C. and subsequently is added of n-pentylchloroformate (0.69 kg, 4.6 mol) at below 10° C. The resulting solution is stirred at less than 10° C. for at least 0.5 hour. After the completion of the reaction, water is added for phase separation. The organic layer is collected and washed with water for about three times. Then organic layer is collected and transferred to a vessel. Then methanol (0.8 kg) is added at below 5° C. Subsequently 25% NaOH (0.8 kg) is added at 0 to 10° C. The resulting solution is maintained at 0 to 10° C. and stirred for at least 0.5 hour. After the completion of the reaction, citric acid (3 kg) is added for quenching the reaction and doing phase separation. The organic layer is collected and the aqueous is continued to wash with methylene chloride. After phase separation, the methylene chloride layer is collected and combined with the previous organic layer. The resulting organic layer is washed with water and the organic layer is collected. The organic layer is concentrated and then is swapped with n-pentanol (3.3 kg) under vacuum at less than 60° C. After solvent swap, n-heptane (0.68 kg) is added and the resulting solution is heated at 40-60° C. and seeded with capecitabine. The mixture is held for about 1 hour at 40-60° C. and cooled down to −5 to 5° C. The slurry is stirred at −5 to 5° C. for about 2 hours. The resulting solid is filtered, washed with n-heptane and dried under vacuum to afford capecitabine (0.9 kg), Yield: about 80%. The purity is ≧99.5%, impurity F≦0.3%, impurity G≦0.2%, impurity H≦0.3%, M2≦0.1%, impurity M≦0.10% and the maximum individual impurity is ≦0.1%.

### Example 7

### Capecitabine Isolation from Mother Liquor of Crystallization

The mother liquor (6 L) of crystallization of capecitabine is added to a vessel. Then the solution is concentrated under vacuum at below 60° C. until the final volume of the residue is about 1 L. The reaction is cooled to 40 to 50° C. (target 45° C.) and seeded with capecitabine. The mixture is held for 1 hour at 40 to 55° C. and cooled to −5 to 5° C. The slurry is stirred at −5 to 5° C. for about 2 hours. The resulting solid is filtered, washed with n-heptane (0.5 kg) and dried under vacuum to afford capecitabine. The purity is 99.5%, the maximum individual impurity is ≦0.1%, water content ≦0.05%. Yield: 10%.

### Example 8

### Synthesis of Capecitabine by Hydrolytic Enzymes-Catalyzed Process

To a suitable reactor of multi-mass reactor is charged compound II (1.0 g, 1 w/w) and co-solvent containing 19:1 of n-BuOH-PPW (20.0 mL, 20 v/w) at room temperature. At this stage the solution is shown clean for stirring 0.5 hr. In another reactor is prepared mixed reagent involved lipase (2.0 g, 2 w/w) and celite (2.0 g, 2 w/w) or silica gel (2.0 g, 2 w/w). Subsequently the mixed solids were charged into the solution for several times and heated to 45° C. after addition. The resulted solution is looked as a slurry mixture. Then IPC monitoring via taking a 50 uL solution into 1 mL ACN, filtered the solid and the filtrate is set into HPLC.

After the completion, BuOH (10 mL, 10 v/w) is added into the solution and the slurry is filtration with Buchner Funnel and dried under vacuum. Collected the solid for the recycle using and the filtrate is concentrated under the vacuum to obtain the crude API.

The invention is not limited by the embodiments described above which are presented as examples only but can be modified in various ways within the scope of protection defined by the appended patent claims.

We claim:

1. A process for making a purified compound of formula (I): 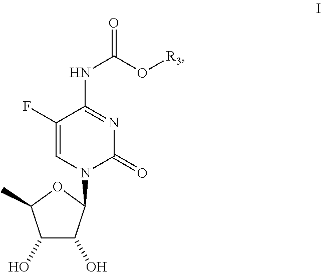
wherein R₃ is alkyl, cycloalkyl, aralkyl, aryl, or alkoxy, comprising:
(a) reacting a compound of the formula (II): 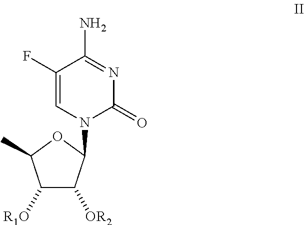
wherein each of R₁ and R₂ independently represents a hydroxyl protecting group, with an acylating agent of formula (III): X—C(═O)—R₃, wherein X is an acyl activating group and R₃ is as defined above, in an organic solvent to produce an acylated compound of formula (IV): 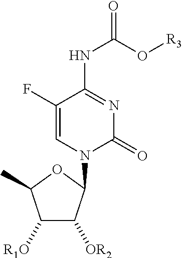
wherein each of R₁, R₂, and R₃ is as defined above;
(b) deprotecting the acylated compound of formula (IV) to obtain the compound of formula (I); and
(c) purifying the compound of formula (I) with a solvent.

2. The process of claim 1, wherein X is halide.

3. The process of claim 1, wherein R₃ is C1˜C6 alkyl.

4. The process of claim 1, wherein R₃ is pentyl group.

5. The process of claim 1 wherein the reacting step (a) is carried out in the presence of a base in an amount from 3.5 to 5.0 mole equivalents of the compound of formula (II).

6. The process of claim 5 wherein the base is pyridine in the amount of 3.5 to 4.5 mole equivalents of the compound of formula (I).

7. The process of claim 1 wherein the deprotecting step (b) is accomplished by a hydrolysis reaction in a temperature of from about 0 to 10° C.

8. The process of claim 1, wherein the solvent is n-pentanol.

9. The process of claim 1 wherein the purifying step c) is carried out at a temperature of less than 60° C.

10. The process of claim 1 wherein the reacting step (a) and deprotecting step (b) are successively carried out in the same reactor.

11. Capecitabine having a mean particle size of D₉₀ is 250 to 350 microns, D₅₀ is 100 to 120 microns and D₁₀ is 25 to 30 microns.

12. A process of making capecitabine, comprising deprotecting a compound of formula (IV) 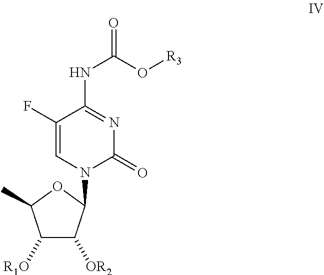
with an enzyme, wherein each of R₁ and R₂ independently represents a hydroxyl protecting group, R₃ is alkyl, cycloalkyl, aralkyl, aryl, or alkoxy.

13. The process of claim 15, wherein the enzyme is lipase.

14. The process of claim 15 wherein R₃ is a pentyl group

15. A capecitabine comprising:
no more than 0.3% by HPLC area percent (A %) of impurity F 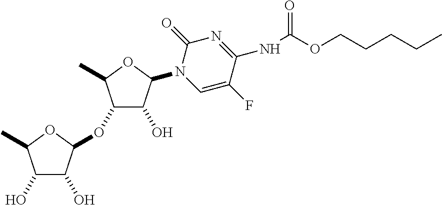
impurity F;
no more than 0.2% by HPLC area percent (A %) of impurity G, 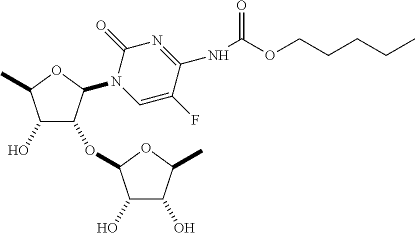
impurity G;
no more than 0.3% by HPLC area percent (A %) of impurity H, 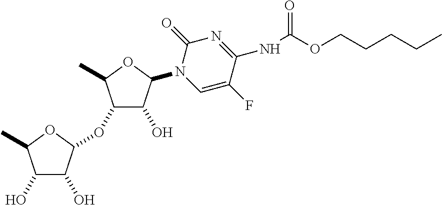
impurity H;
no more than 0.1% by HPLC area percent (A %) of M2, 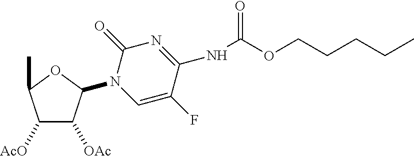
M2; and
no more than 0.10% by HPLC area percent (A %) of impurity M 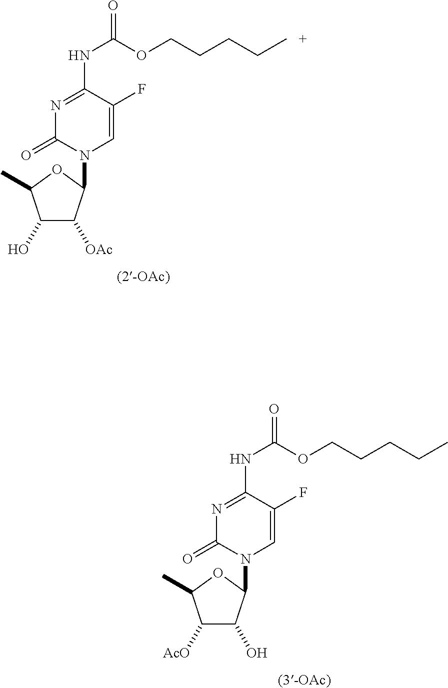
impurity M.
